(19) United States
(12) Patent Application Publication
(10) Pub. No.: US 2009/0281393 A1
(43) Pub. Date: Nov. 12, 2009

(54) Method and apparatus for administering and monitoring patient treatment

(75) Inventor: Remie J. Smith, Mahopac, NY (US)

Correspondence Address: Remie J. Smith, 6 Fox Trail, Mahopac, NY, 10541, US

(73) Assignee: Putnam Technical Group, Inc., Mahopac, NY (US)

(21) Appl. No.: 12/151,700

(22) Filed: May 8, 2008

(51) Int. Cl. A61B 5/00

(52) U.S. Cl. 600/301; 600/300

(57) ABSTRACT

The present invention is a personal health monitoring system which interactively delivers treatment stimuli and compiles a chronological history of a patient's health. The invention includes personal health diaries for direct measurement of physiological measurement as well as collection subjective responses. The personal health diaries also deliver educational content and health related reminders. The personal health diaries are remotely configured to deliver patient specific display of the treatment information. Patient response information, timing signals are routinely transmitted to the central monitoring system. The central monitoring system is programmed to automatically alert caregivers and family members when responses fall outside patient defined normal ranges. Caregivers and family members can interactive with the central monitoring system to provide updated treatment information as well as personal inspirational content. Through education and appropriate intervention the present invention can improve and maintain the patient's health and extend their independent lifestyle.

### BACKGROUND OF THE INVENTION

I. Field of the Invention

The present invention is directed to the field of health care that involves remote monitoring the health status for the general populous with chronic medical conditions. The invention interactively tracks health related developments and filters leading health indicators for acute medical conditions. Through support, education and appropriate intervention the present invention can improve and maintain the patient's health while extending their independent lifestyle. The present invention provides interactive support for the patients through their family, friends and healthcare providers. The present invention includes a plurality of remote monitoring units and at least one central monitoring station. The central station alerts family members, friends and caregivers when the patient's medical condition requires intervention. The central station is connected to a number of monitoring units. The monitoring units are simple, inexpensive, patient operated devices. The physiologic parameters monitored may include but are not limited to blood pressure, pulse rate, blood oxygen saturation, weight, blood glucose, temperature, and pulmonary function.

II. Related Art

Health monitoring systems have been developed in the past which collect information related to the patient's medical condition. Some provide interactive medication regimen support, while others collect subjective and physiologic measurements. No patents teach or disclose a method for health monitoring which include subjective patient measurement, direct patient physiologic measurement, medication compliance, ongoing treatment education and an interactive support network.

Worthington U.S. Pat. No. 3,566,370 discloses a system for the collection patient history using subjective health related questionnaires. The collected information is transmitted to a central monitoring computer system. The Worthington patent does neither teach nor disclose the inclusion of medication compliance nor physiologic measurements.

Allen U.S. Pat. No. 4,731,726 discloses a remote glucose monitoring system which transmits the patients direct physiologic glucose readings to the physician's office. The Allen patent does not teach nor disclose a method for tracking medication compliance, subjective measures, and patient education. Further the Allen patent fails to disclose or teach a method for a support network to enhance the patient's treatment.

Fu U.S. Pat. No. 4,803,625 discloses an interactive device for collecting health related information including medication compliance, subjective and physiologic measurements. However, the Fu patent fails to disclose or teach the interaction with a support network to enhance the patient's treatment and improve the monitor's acceptance into the patient's lifestyle.

Kehr U.S. Pat. No. 5,954,641 discloses an interactive medication compliance device which dispenses medication and provides subjective patients measurements. However, the Kehr patent fails to disclose or teach a method for direct physiologic measurements. Further the Kehr patent fails to disclose or teach a method for a support network to enhance the patient's treatment.

Broas U.S. Pat. No. 6,771,174 discloses an adaptive smart pillbox for medication compliance. The collected information is transmitted to a central monitoring computer system. However, the Broas patent does neither teach nor disclose the inclusion of patient subjective measurement, patient education, nor direct patient physiologic measurements. Further the Broas patent fails to disclose or teach a method for a support network to enhance the patient's treatment.

The foregoing patents have been referenced only by way of general background, because none of them relates to the improvements of this invention. As described in detail below, these improvements enhance the diagnostic value of the system, improve the acceptance of the device into the patient's lifestyle, enhance the patient's lifestyle and reduce the complexity of implementation of the system.

### SUMMARY OF THE INVENTION

The objective of the invention is to improve the lives of people with chronic health conditions while reducing the overall health treatment costs. The invention accomplishes this by providing health monitoring network using personal health diaries configured to present the individual patient's medical regimen. The health diaries are configured to remind patients of their medical treatment needs and elicit responses from the patients to collect subjective and physiological health status information. The patient reminders insure patients follow their medical regimen and the health status information is used to detect early warning conditions. The medical outcomes of people with chronic health conditions can be dramatically improved through adherence to the medical regimen, education, monitoring of key physiologic medical parameters. The major chronic conditions such as Diabetes and Cardiovascular Disease can be successfully managed if early warning signs are detected and acted upon before they progress into more serious stages. Traditionally, these diseases are self managed by the patients and/or by frequent clinical visits. The self management approach results in inadequate treatment and a costly healthcare system with overwhelmed health care resources. In addition, the treatment options for chronic health conditions advances over time as new treatments are introduced. The present invention provides a means to inform and educate patients of these changes. The present invention provides an interactive tool for both caregivers and family. Family involvement plays a key role helping patients adopt the monitoring system into their lifestyle and improving their medical regimen adherence. The resulting system allows the aging population to maintain a healthy independent living condition without expensive medical care.

Remote health care management at a fractional cost of the traditional management approaches. The invention allows participants to effectively manage their condition with appropriate health care intervention when required. The early intervention results in healthy patients, more effective outcomes and an efficient health care system.

Health care management involves six main requirements. A successful effective healthcare management system must fulfill all on these requirements. These requirements are:

### Adherence:

Providing health care requires adherence to medical recommendations and prescribed regimens. Failure to follow medical advice is one of the leading factors in deteriorating health conditions, yet adherence to treatment in general remains at a median rate of approximately 50%ⁱ (Rapoff, 1999). The system must provide customized personal regimen reminders and monitoring to improve the patients outcome. ⁱRapoff, M. A. (1999). Adherence to pediatric medical regimens. New York: Kluwer Academic/Plenum. [58] http://www.dbpeds.org/articies/detail.cfm?TextID=122

### Monitoring:

The system must monitor the patient's condition for both subjective and physiologic conditions. This information must be gather form direct querying of the patient through questionnaires as well as direct measurement of key medical indicators using medical devices (e.g. blood pressure, blood glucose, etc)

### Education:

The treatment options for chronic conditions change over time. New drugs are introduced and improved therapies evolve. The system must successfully educate the users and family as treatment change over time. The system must educate and inform patient with news articles and treatment education.

### Family Involvement:

The involvement of the patient's family is a fundamentally overlooked treatment mechanism. Within the chronic disease population parents and children are often separated by responsibilities and distances making family directed health care management impossible. However, the family members concerns and relationships make them one of the best healthcare management options. By allowing the family members to participate in the health care process the patients outcome can be further improved.

### Low Cost:

There have been numerous medical systems developed to assist in the management patient medical conditions. The vast majority of these systems are too costly to be implemented to the general populous. The present system is targeted at generally healthy patients managing one or more chronic conditions. The personal health diary can be distributed at little cost to the patient population.

### Habitual Acceptance:

A medical system must be used in order to be successful. Most previous systems strictly function as a medical system. When users interact with these systems they are only reminded of their medical conditions the system loses acceptance due to the negative connotation it delivers. The current invention turns to system into a communication portal between the patient and their families. This extension into the personal life of the patient makes it possible to deliver medical advice as well as a rewarding personal experience. This positive experience results in a higher acceptance rate by the users.

### The Disease Management Landscape

As of 2005 the NIH reported that seven percent (20 million) of the population of the United States have diabetesⁱⁱ (NDIC 2005). In addition sixty percent (12 million) of the diabetes have some form of ⁱⁱNational Diabetes Clearing House (NDIC) http://diabetes.niddk.nih.gov/dm/pubs/statistics/ cardiovascular diseaseⁱⁱⁱ (American Heart Association 2007). These diseases have complex treatment regimens involving multiple medications, ongoing education and monitoring of key subjective and physiologic measures.ⁱⁱⁱ Heart Disease and Stroke Statistics-2007 Update http://circ.ahaiournals.org/cgi/content/full/CIRCULATIONAHA.106.179918

The health outcomes of diabetics can be improved through management of blood sugar levels, blood pressure, diet, medication adherence and by receiving other preventive care in a timely manner. Diabetes can affect many parts of the body and can lead to serious complications such as blindness, kidney damage, and lower-limb amputations. The occurrence of these and other diabetes complications can be reduced by controlling the levels of blood glucose, blood pressure, and blood lipids.

### Glucose Control

Studies in the United States and abroad have found that improved glycemic control benefits people with either type 1 or type 2 diabetes. In general, every percentage point drop in A1C blood test results (e.g., from 8 to 7 percent) reduces the risk of microvascular complications (eye, kidney, and nerve disease) is reduced by 40 percent.

### Blood Pressure Control

Blood pressure control reduces the risk of cardiovascular disease (heart disease or stroke) among persons with diabetes by 33 to 50 percent, and the risk of microvascular complications (eye, kidney, and nerve disease) by about 33 percent.

In general, for every 10 mm Hg reduction in systolic blood pressure, the risk for any complication related to diabetes is reduced by 12 percent.

### Control of Blood Lipids

Improved control of cholesterol or blood lipids (for example, HDL, LDL, and triglycerides) can reduce cardiovascular complications by 20 to 50 percent.

### Preventive Care Practices for Eyes, Kidneys, and Feet

Detecting and treating diabetic eye disease with laser therapy can reduce the development of severe vision loss by an estimated 50 to 60 percent.

Comprehensive foot care programs can reduce amputation rates by 45 to 85 percent.

Detecting and treating early diabetic kidney disease by lowering blood pressure can reduce the decline in kidney function by 30 to 70 percent.

The above and other conditions reflect a growing need for ongoing patient monitoring. Some monitors have been developed for recording and transmitting certain patient-related information between remote locations and central stations or physicians, offices.

### BRIEF DESCRIPTION OF THE DRAWINGS

The invention will be understood more fully from the detailed description given below and from the accompanying drawings of embodiments of the invention which, however, should not be taken to limit the invention to the specific embodiments described, but are for explanation and understanding only.

FIG. 1 is a drawing illustrating the important elements of the Health Monitoring Network of the invention including various communication configurations.

FIG. 2 is a drawing illustrating the top level data flow of the invention showing the main users and physical components of the Health Monitoring Network.

FIG. 3 is a drawing illustrating the important elements of the invention and their information flows where solid lines represent communication links and control relationships.

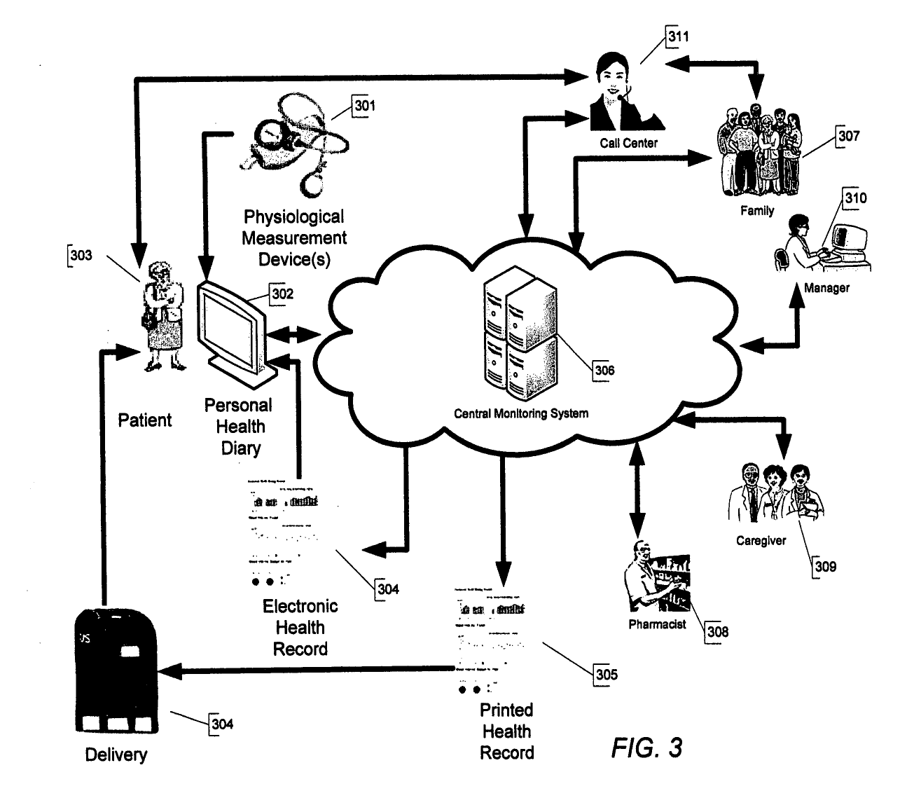

FIG. 4 is a drawing illustrating the significant components of the central monitoring station with communication links and control relationships.

FIG. 5 is a drawing illustrating the significant components of the patient health diary with communication links and control relationships.

FIG. 6 is a drawing illustrating the block diagram illustrating the internal components of the Personal Health Diary.

FIG. 7 is a drawing illustrating the structural layout of flash storage for language and region dependent elements on the Personal Health Diary.

FIG. 8 is a drawing illustrating the rendering of User Interface Markup Language screen templates into user interface screens on the Personal Health Diary.

FIG. 9 is a drawing illustrating the user interface structure with control relationships on the Personal Health Diary.

FIG. 10 is a drawing illustrating the Markup Language Health Regimen Definition on the Personal Health Diary. The illustration shows a sample medication with a single weekly dosage.

FIG. 11 is a drawing illustrating the Markup Language Health Regimen Definition on the Personal Health Diary. The illustration shows a sample questionnaire with three question options.

FIG. 12 is a drawing illustrating the Markup Language Health Regimen Definition on the Personal Health Diary. The illustration shows two sample alerts with scheduling options.

FIG. 13 is a drawing illustrating the Markup Language for recorded data logged events on the Personal Health Diary.

### DETAILED DESCRIPTION OF THE INVENTION

The following description will first express the invention from a system standpoint, then the hardware and finally the software of one embodiment of the invention. All patents, patent applications and literatures cited in this description are incorporated herein by reference in their entirety. In the case of inconsistencies, the present disclosure, including definitions, will prevail.

The physical system is comprised of three essential elements, (1) the health monitoring network, (2) the personal health diary and (3) the central monitoring system. The invention is designed as a personal health monitoring system for automated administration of medical advice to a patient, including communicating with a remote central monitoring station. In general terms the invention is designed (1) to deliver medication regimen information, (2) to prompt patients to respond to questionnaires, (3) record physiological measurements, (4) record subjective responses to questionnaires (5) remind patients to take medication, (6) educate patients relating to their medical condition, (7) log events as the patient responds or fails to respond to delivered prompts, (8) communicate events, responses and measurements to a central monitoring station, (9) alert caregivers and family as critical medical conditions develop (10) create a patient health record which is delivered to the patient.

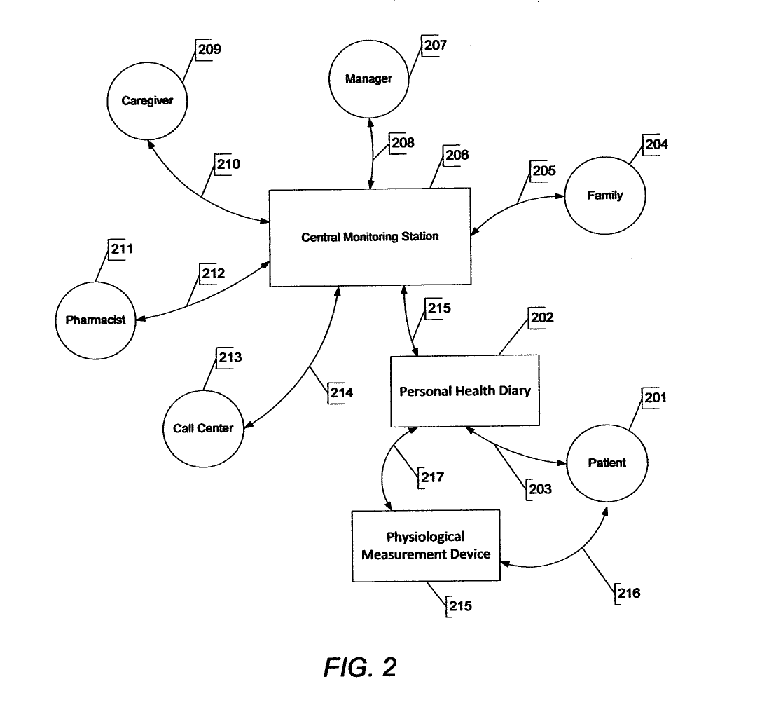

FIG. 2 illustrates a top level data flow diagram showing the main users and physical components of the health monitoring network. The health monitoring network users are divided into six roles. The description of each role and their functions are detailed below:

FIG. 2.201 Patients: The patient user utilizes the personal health diary device (FIG. 2.202) and physiological measurement devices (FIG. 2.215) to interface with health monitoring network. The target patient of the personal health diary are elderly will little or no computer literacy. A sixth grade education and functional level is assumed. The personal health diary alerts patients with chimes and vocal prompts as needed. The personal health diary display prompts patients to respond to questionnaires, prompts patients to use physiological measurement devices (FIG. 2.215), the personal health diary records physiological measurements (FIG. 2.217), records subjective responses to questionnaires (FIG. 2.203) reminds patients to take medication (FIG. 2.203), and educates patients relating to their medical condition (FIG. 2.203). The personal health diary device (FIG. 2.202) communicates (FIG. 2.215) with the central monitoring station (FIG'2.206). In some embodiments of the invention, the personal health diary is connected to the phone line at the patient user's location. Based on the personal health diary device configuration, at a specified time, the device will dial (FIG. 2.215) the central monitoring station (FIG. 2.206). The personal health diary device will then upload results and download new configuration settings as required. Periodically, health records are sent to the patient and caregivers via electronic and corporeal delivery mechanisms.

FIG. 2.204 Family: Family members provide support for the patients. Family members receive alert reports when patients report adverse medication events (FIG. 2.205). Typically these would include repeatedly missing medications, signs of depression, and reports of medication side effects. In addition Family members carl submit event requests to the central monitoring station. These requests are used to deliver patient specific content. Typically this includes personal content such as birthday reminders, pictures, short video messages and similar motivational content. These requests are reviewed by the caregiver, approved and posted for delivery to the patient's regimen. As needed family members can communicate with the call center to update patient specific health information;

FIG. 2.207 Manager: The manager will manage the creation of treatment templates. This role has read/write access to all treatment pattern information on a central monitoring station. The manager interacts with the central monitoring station to define and create questionnaires, education materials, motivational content and alert definitions (FIG. 2.208);

FIG. 2.209 Caregiver: The caregiver oversees a set of patients enrolled on the central monitoring station. The caregiver interacts with the central monitoring station to (1) enroll patients, (2) monitor treatment, (3) review and sign the event data, (4) manage alerts, (5) approve motivational multimedia content and (6) closeout the patient involvement (FIG. 2.210). Caregivers receive alert reports when patients report adverse medication events (FIG. 2.210);

FIG. 2.211 Pharmacists: Pharmacists fill prescriptions for patients and review medication dosing schedules (FIG. 2.212). Pharmacists receive alert reports when patients request medication refills or report medication side-effects;

FIG. 2.213 Call Center: The call center will communicate directly with patients and other users. Depending on the training level of the call center personnel they may also have caregiver or manager user privileges. The call center interacts with the central monitoring station to log incoming calls (from patients, other users), triage requests and find resolutions (FIG. 2.214). In the event the call center cannot resolve the problem they can reassign the problem to other users. Other users will be notified via alert reports;

### Health Monitoring Network

The health monitoring network is comprised of remote personal health diaries that are networked to remote central monitoring stations. In the present preferred embodiment the personal health diaries utilize a wireless Bluetooth communication network to interface to Bluetooth enabled Public Switched Telephone Network (PSTN) MODEMS. In this configuration personal health diaries can be placed in a location convenient to the patient's lifestyle without concern to the location of a telephone interface jack.

The communications schema utilizes toll free dialup services to the remote central monitoring station. The personal health diaries are programmed to dial the central monitoring station during overnight hours. Additionally, the personal health diaries are programmed to recognize critical health information. Any critical information is communicated soon after it is recorded.

The personal health diaries are programmed to randomize the dial in times and randomized dial in retries periods to distribute network load. In the event of a communication failure, the personal health diaries will repeat communication attempts until a successfully completing a communication session. The network is equipped with redundant Public Switched Telephone Network (PSTN) services, toll free and remote access servers to improve network reliability.

In one embodiment, the toll free services directly dial into a remote access services on the remote central monitoring station. The local Public Switched Telephone Network (PSTN) services are programmed with rollover capabilities this enables the MODEM bank configured on the central monitoring station.

The present communications schema provides a highly integrated, efficient method and structure of transferring personal health information. Other embodiments of the invention the personal health information can be transferred on various networks in which various networks such as Cable Television Network, Local area Network (LAN), a wide area network (WAN) Integrated Services Digital Network (ISDN), the Internet, a wireless network, an asynchronous transfer mode (ATM) network, fiber optic networks, satellite, mobile and other similar networks are implemented to transfer voice, data and video between the personal health diaries and a central monitoring stations. In the preferred embodiment, Public Switched telephone Network (PSTN), the Internet, and wireless networks are illustrated as examples only and should be viewed without limiting the invention to these types of communications alone. Further, in the interest of simplicity, the applicants reference to the various communications system, in relevant parts, as a communications system. However, it should be noted that the communication systems, in the context of this invention, are interchangeable and may relate to various schemes of cable, fiber optics, microwave, radio, laser and similar communications or any practical combinations thereof.

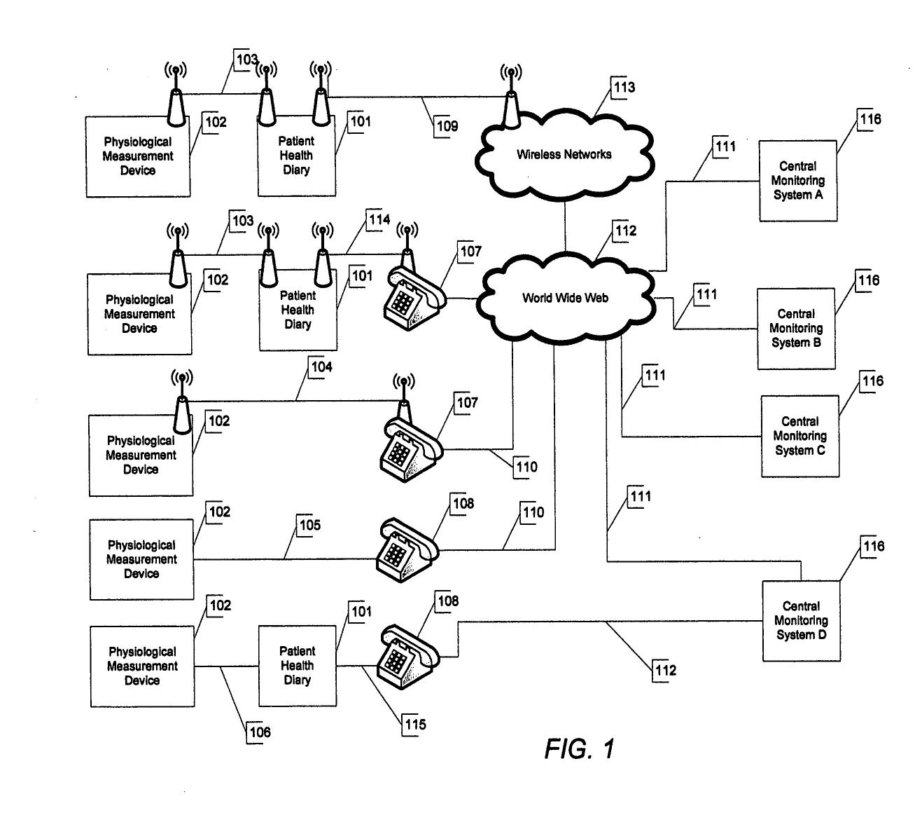

FIG. 1 is a block diagram of the personal health monitoring network; the figure illustrates the invention, a personal health monitoring system for automated observation of medical conditions of patient users, including communicating with a remote central monitoring station.

FIG. 1 illustrates the personal health diaries (FIG. 1.101) connected (FIG. 1.115) to hardwired MODEMS (FIG. 1.108), connected (FIG. 1.114) to wireless MODEMS (FIG. 1.107), and connected (FIG. 1.109) to wireless LANS/WANS (FIG. 1.113);

FIG. 1 further illustrates the MODEMS (FIG. 1.107, FIG. 1.108) connecting (FIG. 1.110) to the World Wide Web (FIG. 1.112) or directly connecting (FIG. 1.112) into a central monitoring station (FIG. 1.116);

FIG. 1 further illustrates various physiological measurement devices (FIG. 1.102) connected to the health monitoring network. The physiological measurement devices (FIG. 1.102) are shown connected (FIG. 1.106) using hardwire or using connect (FIG. 1.103) wirelessly to the personal health diaries (FIG. 1.101). In addition physiological measurement devices (FIG. 1.102) are shown connected (FIG. 1.105) using hardwired MODEMS (FIG. 1.108), and shown connected (104) using wireless MODEMS (FIG. 1.107);

FIG. 1 further illustrates multiple central monitoring stations configured on the network. Central monitoring stations (FIG. 1.116) can be configured to connect to the World Wide Web (FIG. 1.111). Several central monitoring stations (FIG. 1.116) can be configured for redundancy or to service separate patient populations.

### Personal Health Diary

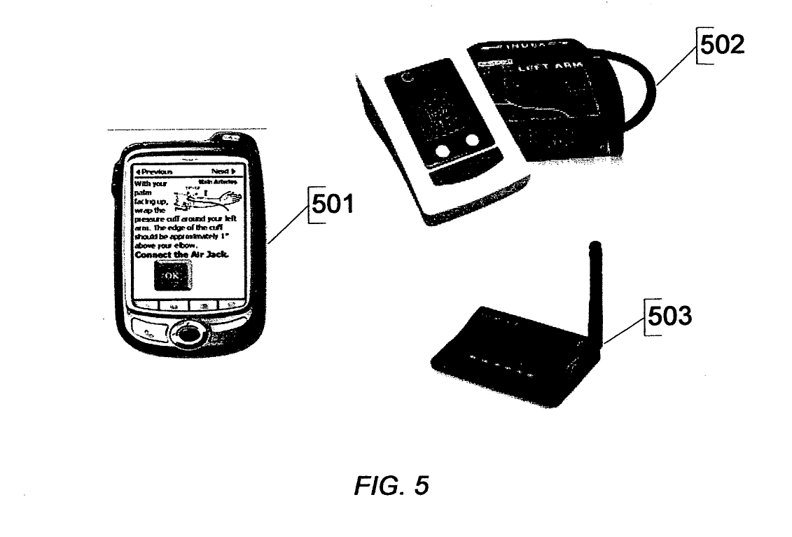

The personal health diary consists of custom programmed electronic device. FIG. 5 illustrates three typical components for a patient on the personal health monitoring network. Apparatus FIG. 5.501 illustrates the personal health diary. The personal health diary is a microprocessor controlled unit which executes the personal health diary software. In one embodiment a Dell Axim x51 PDA (Dell Computer Corporation, Round Rock, Tex.) serves as the personal health diary. The apparatus FIG. 5.502 illustrates wireless medical devices used to collect patient health measurements. In one embodiment a Taidoc TD-3258 (TaiDoc Technology Corporation. Wugu, Taipei County 248, Taiwan) is used to collect both blood pressure and blood glucose readings from the patients. Lastly, apparatus FIG. 5.503 illustrates a modem used to interface personal health diary (FIG. 5.501) to the patient home phone system. In one embodiment a Zoom Bluetooth MODEM model 4300 (Zoom Technologies, Boston Mass.) is used.

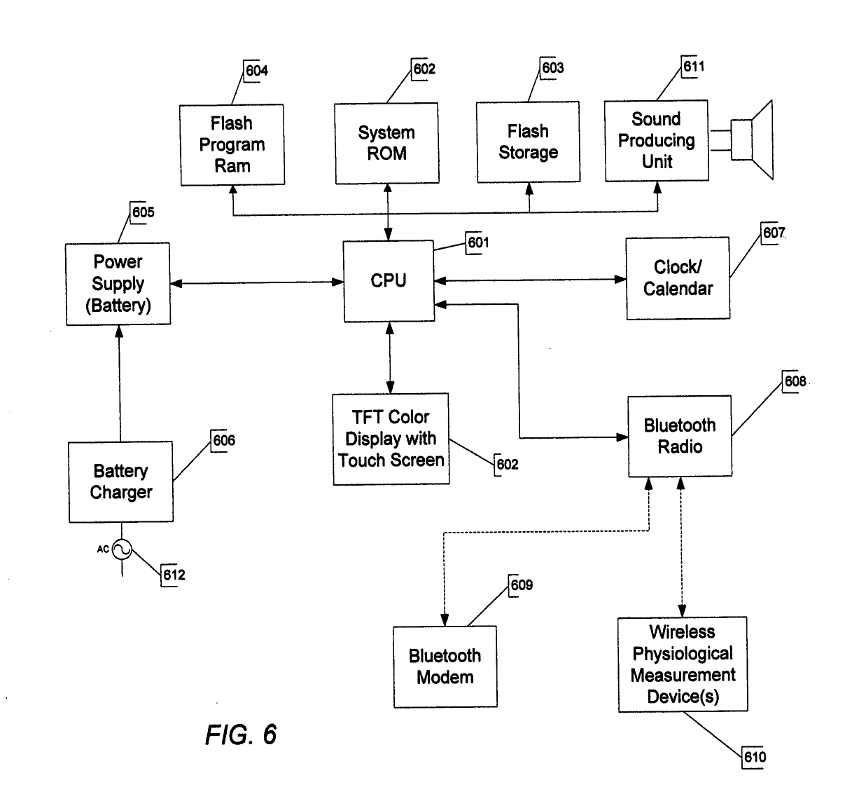

FIG. 6 depicts apparatus FIG. 5.501 in greater detail. The Microprocessor (CPU) (FIG. 6.601) is interfaced to several system components. The basic operating system is programmed into the system Flash ROM (FIG. 6.602). The personal health diary software is stored in non-volatile Flash Program RAM (FIG. 6.604). Patient Medical regimen information, logged events, education materials and user interface templates are stored in Flash Storage unit (FIG. 6.603). The advantage of Flash Program Ram (FIG. 6.604) and Flash Storage (FIG. 6.603) is the unit will retain all program and configuration data if Power Supply (Battery) (FIG. 6.605) is completely drained. The Microprocessor (CPU) (FIG. 6.601) interfaces to a clock calendar chip set (FIG. 6.607) to provide accurate timing and scheduling for the patients regimen. The Microprocessor (CPU) (FIG. 6.601) interfaces to a Sound Producing unit (FIG. 6.611) to provide both alerts and voice instructions. The Microprocessor (CPU) (601) interfaces to TFT Color Display with touch screen to present instructions and education information to the patient user. The Microprocessor (CPU) (FIG. 6.601) interfaces to Bluetooth Radio chipset (FIG. 6.608). The Bluetooth radio is used to interface to a Bluetooth MODEM (FIG. 6.609) as well as Physiological Measurement Devices (FIG. 6.610). The Microprocessor (CPU) (FIG. 6.601) interfaces to Power Management Chip set to monitor battery power and battery charging. Battery charging is provided by an external charger (FIG. 6.606) which is in turn connected to AC power (FIG. 6.612). The personal health diary in one embodiment utilizes Microsoft's Windows CE® (Microsoft Corporation, Redmond, Wash.) as the core operating system. This selection was made since the operating system is open on many mobile devices allowing customized software to be written and ported to a wide variety of devices. The operating system supports a wide variety of display and input devices. In one embodiment, using the Dell Axim X51, a touch screen interface with a 240 by 320 pixels capable of displaying 64535 colors (QVGA) provides a small portable device with a high resolution display screen. The touch screen is utilized to allow patients to directly input responses on the screen. The personal health diary also supports battery powered operation. In this one embodiment the personal health diary is a pocket size apparatus allowing for fully portable usage.

The customized software on the personal health diary is implemented to hide the complexity of the operating system operation. The software replaces the operating system user interface with an interface dedicated to the personal health diary operation. The software supports multiple languages, locales allowing the invention to be utilized worldwide. The software user interface utilizes color, graphics, multimedia (including sound, voice and video) to present instructions in a clear fashion. The communications with the central monitoring system is completely transparent to the users. In one embodiment the personal health diary software communicates with a wireless Bluetooth modem. Yet another embodiment utilizes a cellular wide area network for communications. The personal health diary is simple to setup requiring the patient or caregiver to connect the personal health diary to AC power and connect the modem to a PSTN phone connection and AC power. The Physiological Measurement Devices are typically battery powered for safety reasons. The personal health diary software is implemented to meet all regulatory requirements.

Since the personal health diary presents information on a scheduled routine it is required that the device maintains accurate time. Time synchronization is achieved when the device communicates with the central monitoring station. The personal health diary allows for selection of current time zone regions to allow for proper display of local time, handling of daylight savings and facilitate patient travel through regional time zones.

The personal health diary software application is implemented as an object oriented application. The software application class structure is depicted below:
Application Level
   User Interface Layer (UI)
   Business Application Layer (BAL)
   Data Application Layer (DAL)
   Common Application Layer (Common)

The Application Level is the main entry point for the software application. The user interface is directly implemented under this level. The user interface communicates with the business application layer (the BAL). The BAL is responsible for implementation of all functional application logic; this includes scheduling, regimen interpretation, alarms and application state. The BAL Layer interfaces to the Data Application Layer (the DAL). The DAL is responsible for data storage/retrieval and communication with the central monitoring station. The DAL Layer implements CRUDE (Create, Retrieve, Update, Delete, and Enumerate) data access functions. The Common Application Layer (Common) provides application utility functions which are shared across all the application layers.

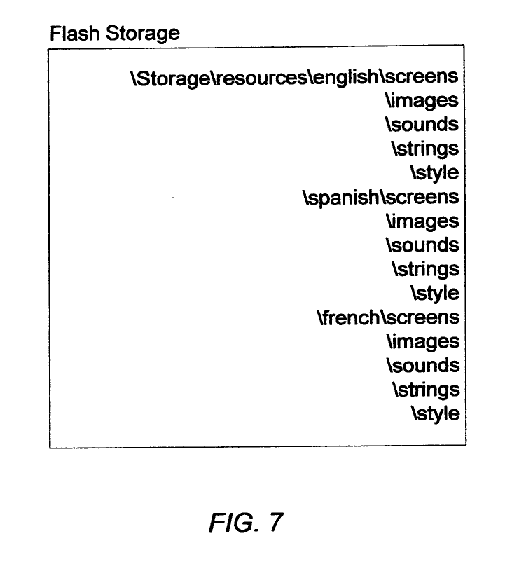

The User Interface Layer is implementation separates the screen layout and format from the program logic needed to display the information. This allows for flexible implementation of language and cultural specific displays. FIG. 7 illustrates how the markup-language user interface utilizes the Flash Storage on the file system on the personal health diary. This permits support of multiple languages for the software application using the file system folder structure. The folder structure on FIG. 7 illustrates the layout of user interface elements for English, Spanish and French. The invention can be further expanded to represent any number of languages or user interface layout preferences.

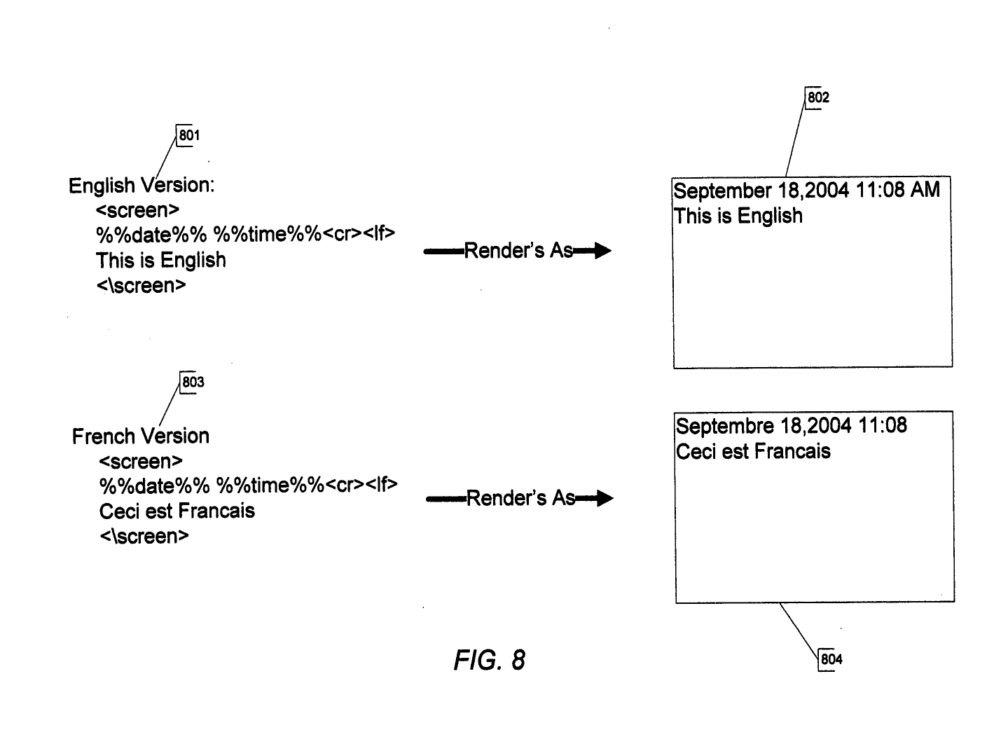

FIG. 8 illustrates the user interface screens are expressed in the user interface markup-language which is external to the compiled application. The user interface screens are expressed in a TAGGED format. The TAGGED format is expressed with starting and ending tags as <TAG> expression</TAG>. The application defines a variety of tags for expressing layout, interactive controls, runtime values, and graphics, multimedia and formatting. The markup-language is rendered by the software application as a graphic screen representation. The rendering software has a secondary set of runtime replaceable tags. The secondary tags are delineated with double percentage characters (e.g. ‘%%time%%’). In the prior example the tag ‘%%time%%’ is used to represent a runtime replaceable time value. The software application will search for tags and replace tags with runtime values prior to rendering the user interface screen. FIG. 8 illustrates an English Language markup-language screen definition (FIG. 8.801) and the rendered graphic screen representation (FIG. 8.802). FIG. 8 illustrates a French Language markup-language screen (FIG. 8.803) and the rendered graphic screen representation (FIG. 8.804). The rendering software and markup-language supports a recursive rendering method allowing for nested sets of replaceable tags.

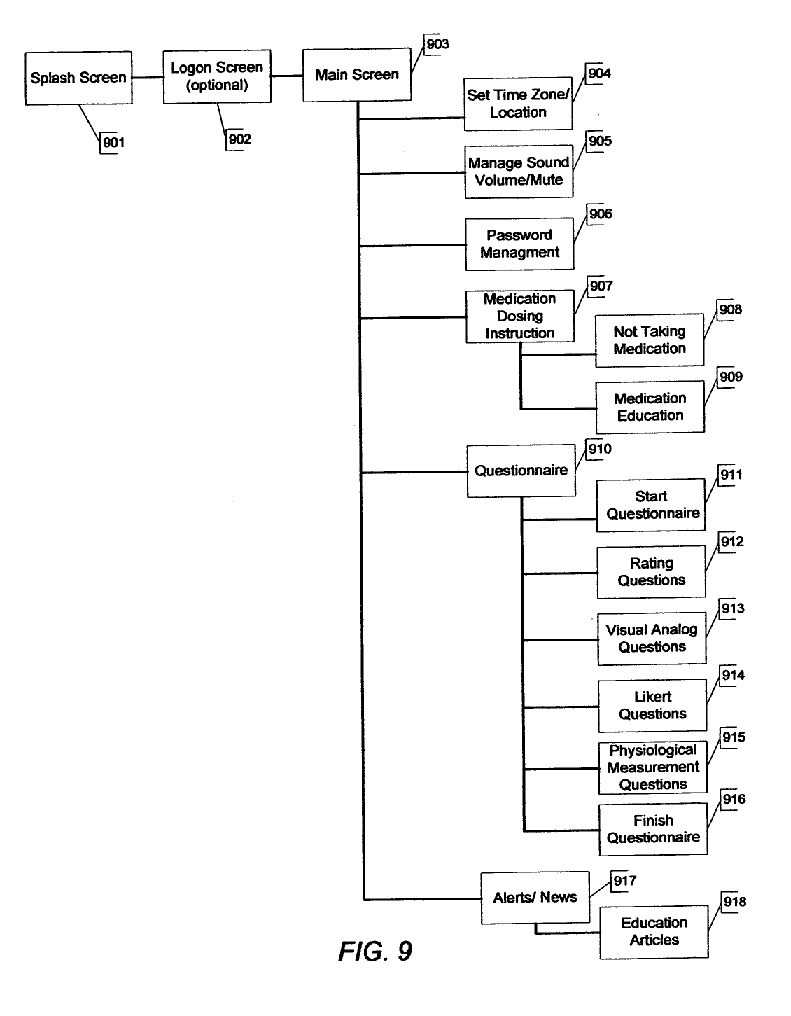

FIG. 9 illustrates the user interface screen map for one embodiment of the personal health diary. Upon startup the software displays a Splash Screen (FIG. 9.901) which displays a customized graphic image for the personal health diary. This screen is displayed while the software loads and initializes all the remaining components. If configured the software will then display a logon screen (FIG. 9.902) which is intended to protect access to any personal health information and confirm the user's identity before continuing. The software then displays a main screen (FIG. 9.903) which displays the status of the personal health diary. The main screen (FIG. 9.903) will display pending health related items the user need to accomplish. Pending health related items appear as prompts which the user clicks on each item to present further instructions. The personal health diary may display reminders for pending medication dosages, reminders to record physiological measurements, reminders to record subjective responses to questionnaires and reminders to review education and news articles relating to their health condition. In addition the personal health diary may be programmed to emit recorded tones or voice instructions to call attention to the pending health related items.

### Medication Reminders

When a patient clicks on a pending Medication Dosage a specific medication dosage instruction screen (FIG. 9.907) is presented. The screen may have a generic reminder to take medications or may prompt with specific medication. When prompting for specific medication the prompt may include specific doses instructions, images and education to further assist the patient. The patient then clicks an ‘OK’ button to indicate they have taken the medication. The patient may also review detailed medication information by clicking on a ‘More Information’ prompt. Clicking on ‘More Information’ will display screen (FIG. 9.909) which contains the information which would be displayed on a drug information sheet for the specific medication. If the patient cannot take the dosage they can so indicate by press the ‘I Can't’ button. Pressing ‘I Can't’ will in turn display the ‘Problem Taking Medication’ screen (FIG. 9.908). The ‘Problem Taking Medication’ screen will allow the patient to indicate a reason for not taking a dosage.

### Questionnaires

When the patient clicks on pending questionnaires the start questionnaire instruction screen (FIG. 9.911) is presented. The screen has both an ‘Ok’ continue button and a ‘Later’ cancel button. Questionnaires are configured on the central monitoring system. The questionnaires contain a series of questions which elicit health related information from the patient. The Questionnaires can be designed to branch based on the patient's responses. Specific question responses will cause the personal health diary to jump to other questions or halt the questionnaire. In addition questionnaire can be configured with questions that initiate communication sessions. These communication enabled questions allow the personal health diary to immediately transfer urgent health related information. Each question in the questionnaire has a previous and next question option. The previous and next question option allows patients to review previously answered questions and change their response. The personal health diary supports several types of questions. The first type a ‘Rating Question’ presents the question text followed by possible responses. The responses are presented as multiple choice options which the user may click on to select (FIG. 9.912). The second question type is a ‘Visual Analog Scale’ (FIG. 9.913). The visual analog scale questions present the question text and a graphic bar with indexed values. The user can move a pointer on the graphic bar to select an appropriate value relating to the question text. A visual analog scale can be used to input health related measurements such as weight or to input values which represent subjective responses regarding the patient's condition. A derivation of the two question types (rating and visual analog scale) is the “Likert Scale”^{iv} (FIG. 9.914) the Likert scale can be either a rating type question or visual analog question in which patients indicate their agreement or disagreement with objective criteria. For example a visual analog scale could present the text “On a scale of 0 to 10 please indicate the current pain you are experiencing”. In this example the graphic bar would then be indexed with values from zero (0) to ten (10) allowing the user to select the value which best represents their current pain level. The personal health diary can also be configured to collect direct physiologic measurements (FIG. 9.915). These physiologic measurement questions directly interface to a hardware device which measures the value. The values are then transferred into the personal health diary. The physiologic measurement questions present a series of instructions guiding the patient through collection of the measurement. Once patient responses to the final question in the questionnaire the personal health diary presents the ‘Finish Questionnaire’ (FIG. 9.916) screen. The Finish Questionnaire screen allows patients the final opportunity to review question responses and change them as needed. Likert, Rensis (1932), “A Technique for the Measurement of Attitudes”, Archives of Psychology 140: pp. 1-55

### Alerts/News Articles

When the patient clicks on pending alert/news article the Alerts/News screen (FIG. 9.917) is presented. The screen presents a headline for the alert/news article and has a confirmation button. The patient may also review detailed information by clicking on a ‘More Information’ prompt. Clicking on ‘More Information’ will display screen (FIG. 9.918) which contains detailed information relating to the alert/news article.

### Patient Configurable Options

There are a set of options on the personal health diary which the patient may change to fit their lifestyle. Currently, these options include the ability to set the volume of personal health diary alarms and voice prompts (FIG. 9.905). In addition the patient may be in a situation when they cannot immediately address the alerts. In these situations the patient can mute the personal health diary, the device will stay muted until an alert reaches a critical phase. In specific patient regimen configurations the personal health diary may be configured to authenticate the user's identity, this feature will hide personal health information until the user identity is confirmed. The user may select a password for the personal health diary; password selection is configured via the password management screen (FIG. 9.906). The patient can also set their current time zone (FIG. 9.904). The personal health diary allows for selection of current time zone regions to allow for proper display of local time, handling of daylight savings and facilitate patient travel through regional time zones.

### Regimen Markup Language

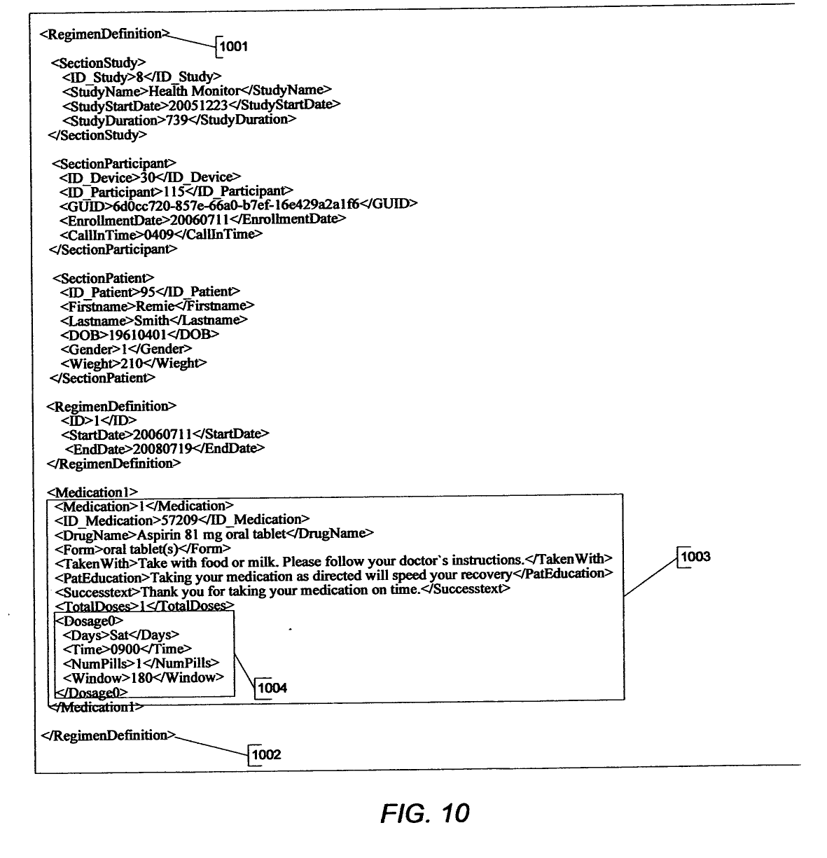

The personal health diary is remotely configured with a patient specific healthcare regimen. The regimen definition is expressed in a markup language which defines the scheduled delivery, definition information, instructions and prompts for each patients care. The regimen is expressed as a series of markup language tags. Each tag set defines a different aspect of the patient healthcare definition. FIG. 10 illustrates a regimen fragment highlighting the sample structural features of the markup language. Each markup-language tag has a well structured starting tag (FIG. 10.1001) and ending tag (FIG. 10.1002). Each set of markup-language tags defines an aspect of the healthcare regimen. FIG. 10.1003 illustrates a sample medication definition. FIG. 10.1004 depicts a nested set of dosage tags within the medication definition. The regimen markup language of the current invention is not limited to the tags shown markup language can be expanded to accommodate new tags as needs arise. The structure of the regimen markup language schema is flexible in that new tags sets can be added without altering the definition of pre-existing tags. This allows additions of new features without altering the behavior of previous versions. This is accomplished by the regimen parsers ability to ignore new unknown tags. The regimen markup language supports tags for scheduling events within the regimen definition. There are two tags for event scheduling, the <Time/> and <Days/> tags. The <Time> tag is used to specify inter-day timing of events. The currently supported formats are as follows: 1) Fixed times of day are expressed in military format such as 0900=9:00 AM or 2100=9:00 PM. 2) The system can also accept special commands such as a time series e.g. ‘0900,1200,1500’, user invoked e.g. ‘Menu, “Initial Survey”’ or As Needed Scheduling e.g. ‘PRN,4,“Glucophage 750XR”’. The <Days> tag is used to specify intra-day occurrences. If the <Days> tag is omitted the event is assumed to occur every day. The <Days> tag has a number of permutations for example “Every 30” would schedule an event to occur every thirty days, “Mon,Wed,Fri” would occur every Monday, Wednesday and Friday, ‘20041027’ would schedule an absolute date of Oct. 27, 2004. The current invention supports several other syntaxes for both the <Time> and <Days> markup-tags. It will be recognized by those skilled in the art that various other types of scheduling can be accomplished in the embodiments described herein without departing from the scope and the spirit of the invention.

The regimen markup language may contain medication dosing instructions. There are two basic dosing forms supported by the current embodiment of the invention, these are outlined below:
1. Scheduled: The personal health diary will prompt the patient to take medication at a specified date and time. This form of scheduling will include day/date and time information. The scheduling may also include time spans and priority alarming information.
2. As Needed Scheduling: The personal health diary also supports “as needed” scheduling. This scheduling allows the patient to take medication doses “As Needed” while logging the information that the dosage was dispensed. This is also known as “PRN” dosing. These as needed doses include a repeat dosing interval to prevent patients from overdosing on as needed scheduling.

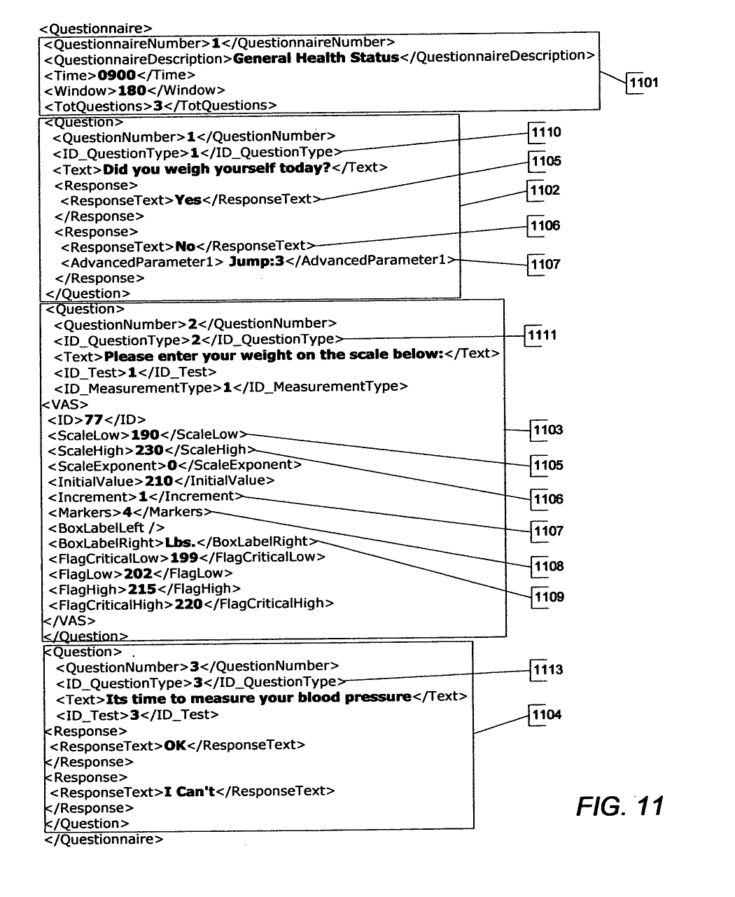

The regimen markup language may contain one or more questionnaires. FIG. 11 illustrates a regimen fragment with a simple three question questionnaire. Label 11.1101 illustrates the header for a questionnaire named “General Health Status”. This example header shows a daily scheduling of 9:00 AM and a total of three questions. The personal health diary supports different question types. Label (FIG. 11.1102) depicts a rating question. The markup tag <ID_QuestionType> with a value of 1 (FIG. 11.1110) defines the question as a rating type question. The rating question contains the question text “Did you weigh yourself today?”. The possible responses to this question are “Yes” (FIG. 11.1105) or “No” (FIG. 11.1106). Note that the response “No” contains a branching instruction (FIG. 11.1107) to question 3 (FIG. 11.1104). The second question in the questionnaire illustrates a visual analog scale question (FIG. 11.1103). The markup-tag <ID_QuestionType> with a value of 2 (FIG. 11.1111) defines the question as a visual analog scale type. The visual analog scale question contains the question text “Please enter your weight on the scale below:”. The visual analog scale is defined with a lower bound of 190 (FIG. 11.1105) and an upper bound of 230 (FIG. 11.1106). The visual analog scale moves in increments of 1 (FIG. 11.1107) allowing users to make selections from 190 to 230 pounds. The scale is labeled in pounds (Lbs.) with the <BoxLabelRight> markup-tag (FIG. 11.1109). In addition the scale is divided by four visual reference tick marks as indicated by the <Markers> markup-tag. The third question (FIG. 11.1104) in the questionnaire is a direct physiologic measurement as indicated by the markup-tag <ID_QuestionType> with a value of 3 (FIG. 11.1113). This question is measuring blood pressure as indicated by the markup-tag <ID_Test> with a value of 3. When a patient selects the first response <responsetext> “OK”; the personal health diary will lead the patient through collecting a blood pressure value.

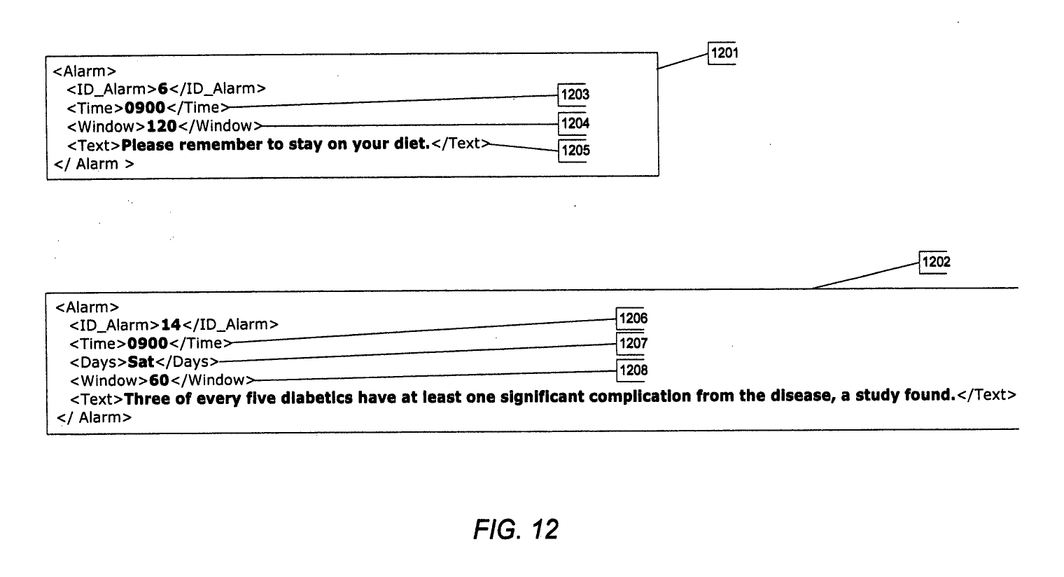

The regimen markup language may contain one or more health related education articles, news articles and personal inspirational content. FIG. 12 illustrates a regimen fragment with two health related articles. FIG. 12 (FIG. 12.1201) depicts a health related education article with a scheduled reminder at 9:00 AM every day. The reminder defines a two hour compliance window via the markup-tag <Window> with a value of 120 minutes. The markup tag <Text> (FIG. 12.1205) defines the headline text which will appear on the personal health diary. The second health related article (FIG. 12.1202) defines a news type article which is scheduled to remind at 9:00 AM (FIG. 12.1206) on Saturday mornings (FIG. 12.1207). The news article will be available with a compliance window of 60 minutes as defined by the markup tag <Window>. The markup-tag <Text> (FIG. 12.1208) defines the headline text which will appear on the personal health diary for the news article (FIG. 12.1202).

Regimens are transferred from the remote monitoring station to the personal health diary using the health monitoring network described above. All communications with the server are in encrypted form to protect patient information. In one embodiment of the invention the personal health diary dials a modem bank on the remote monitoring station and access the system through a remote server access protocol. Using network access provided through the remote server access protocol the personal health diary authenticates itself to the server, downloads any regimen changes, downloads any related multi-media, and synchronizes its internal real time clock with the remote monitoring station. The patient's regimen is transmitted over the data link is the markup-language format. In one embodiment of the invention the personal health diary utilizes the Microsoft SOAP (Simple Object Access Protocol) to interface with the remote monitoring station. Once the regimen has been transferred it is stored in the personal health diaries flash storage (FIG. 6.603). Upon software startup, the personal health diary loads the regimen from the flash storage (FIG. 6.603). The regimen is parsed when the software loads or during a daily reset. The parsing process builds a set of events for the current day. It will be recognized by those skilled in the art that various other types of data transfer can be accomplished and, in addition, that numerous other changes can be made in the embodiments described herein without departing from the scope and the spirit of the invention.

### Personal Health Diary Events

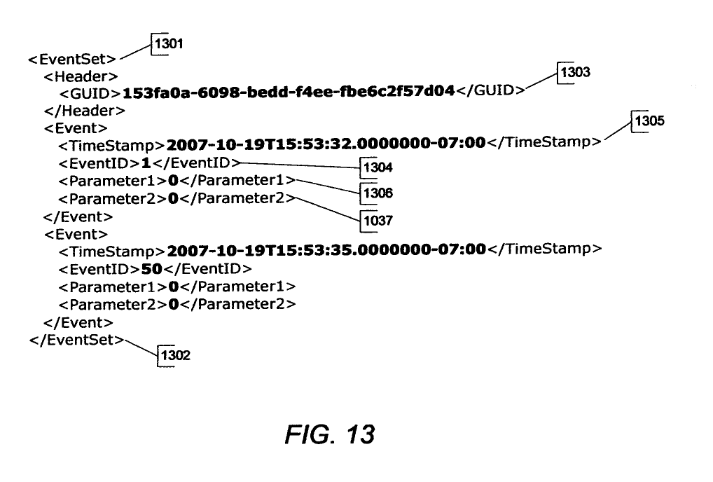

As the personal health diary operates, events are logged based on the regimen configuration. The personal health dairy logs events completed (successful) and events missed (unsuccessful) in an Event Log. The Event Log is expressed in a Markup-language which defines an occurrence timestamp, event type descriptor and related event parameters. The Event Log is expressed as a series of markup-language tags. Each tag set defines a different aspect of the patient's interaction with the personal health diary. FIG. 13 illustrates an Event Log sample highlighting the structural features of the markup-language. Each markup-language tag has a well structured starting tag (FIG. 13.1301) and ending tag (FIG. 13.1302). The Event Log contains a markup-language tag <GUID> (FIG. 13.1303) which uniquely identifies the events for a specific patient regimen. Each set of markup-language tags defines an aspect of an event. FIG. 13 (FIG. 13.1304) illustrates a sample event type descriptor. FIG. 13 (FIG. 13.1305) illustrate the timestamp for an event. FIG. 13 (FIG. 13.1306, FIG. 13.1307) illustrate parameters further characterizing the event.

The Event Log is stored personal health diary in the flash storage (FIG. 6.603). Event Logs are transferred from the personal health diary to the remote monitoring station using the health monitoring network previously described. In one embodiment of the invention the personal health diary dials a modem bank on the remote monitoring station and access the system through a remote server access protocol. The Event Logs are routinely transferred to the remote monitoring station during overnight communication sessions. However, if the Personal Health Diary detects an urgent health situation it will immediately initiate a communication session with the remote monitoring station. Using network access provided through the remote server access protocol the personal health diary authenticates itself, uploads the Event Log to the remote monitoring station. The Event Log is transmitted over an encrypted link in the markup-language format. In one embodiment of the invention the personal health diary utilizes the Microsoft SOAP (Simple Object Access Protocol) to interface with the remote monitoring station. Once the Event Log has been transferred it is cleared in the flash storage (FIG 6.603). It will be recognized by those skilled in the art that various other types of data transfer can be accomplished and, in addition, that numerous other changes can be made in the embodiments described herein without departing from the scope and the spirit of the invention.

### Multimedia Content

The personal health diary is remotely configured with a patient specific multimedia content. The personal health monitoring system can only be successful if the user is willing to use the personal health diary and recognizes its overall contribution to their lifestyle. Previous inventions (see Prior Art) have monitored clinical aspects of the patient's medical condition. These inventions fall short in the long term because they fail to become a habitual part of the patient's lifestyle. Over time patients can grow weary of the redundant medical condition reminders. To overcome this shortcoming the introduction of personally tailored multimedia content keeps the health monitoring system content fresh and relevant in the patient's lifestyle. The present invention allows remote monitor system managers, caregivers and family members to actively participate on the patient's treatment. In one embodiment of the invention the personal health diary provides playback of educational videos, family pictures, family video messages, voice mail messages, electronic letters, electronic greeting cards and holiday reminders.

Multimedia content is stored in the flash storage (FIG. 6.603). Multimedia content is transferred from the remote monitoring station to the personal health diary using the health monitoring network previously described. In one embodiment of the invention the personal health diary dials a modem bank on the remote monitoring station and access the system through a remote server access protocol. Using network access provided through the remote server access protocol the personal health diary authenticates itself to the server, queries the remote server for available multimedia content. The personal health diary then compares its currently stored multimedia content to determine which content can be erased local flash storage and which new content should be downloaded from the remote monitoring station. The patient's multimedia content is transmitted over an encrypted link in the native media format. In one embodiment of the invention the personal health diary utilizes the Microsoft SOAP (Simple Object Access Protocol) to interface with the remote monitoring station. The synchronization and transfer of Multimedia content is automatically accomplished during the overnight communication session. A further advantage to this design is it allows for high quality multi-media content to be displayed on the personal health diary while maintaining a low cost, low speed dial up modem connection. The patient's regimen determines when multimedia content alert reminders are displayed on the personal health diary. When the patient clicks on a content enabled alert reminder they can click on a button to dismiss the reminder or click on ‘More Information’ to view the local multimedia content. In addition in one embodiment of the invention the patient user can also browse the available multimedia content and display or replay it at their convenience. It will be recognized by those skilled in the art that various other types of data transfer can be accomplished and, in addition, that numerous other changes can be made in the embodiments described herein without departing from the scope and the spirit of the invention.

### Central Monitoring Station

The central monitoring station fulfills several system functions in the personal health monitoring network. The central monitoring station delineates functions by user types. Each user may be configured with multiple roles. The functionally of each user is outlined below:

The Manager users (FIG. 2.207) setup treatment programs, this includes configuring the treatment program regimens, questionnaires and medication definitions. The treatment program setup includes scheduling delivery of questionnaires, education, news articles and multi-media content to the treatment program participants. In addition the manager can configure the central monitoring station to deliver alerts to caregivers, pharmacists and family members when specific medical conditions occur.

The Caregiver users (FIG2.209) enroll patient participants and monitor patient care. The Caregiver customizes the patient's regimen to fit the patient's lifestyle this includes time of day scheduling and specific medication reminders. When a Caregiver receives an alert from the personal health monitoring network they can coach the patient or advise them to seek other medical care.

The Pharmacist users can receive alerts from the central monitoring station when specific medication events occur. Typically, a Pharmacist is alerted when a patient reports a medication side effect or needs to refill their medications. When a Pharmacist receives an alert from the personal health monitoring network they can coach the patient or advise them to seek other medical care.

The Family members can receive alerts from the central monitoring station when certain medication specific alerts occur. Typically these would include repeatedly missing medications, signs of depression, and reports of medication side effects. When Family members receive an alert from the personal health monitoring network they can coach the patient or advise them to seek other medical care. In addition Family members can submit event requests to the central monitoring station. These requests are used to deliver patient specific content. Typically this would include motivational content such as birthday reminders, pictures, short video messages and similar content. These requests are reviewed by the caregiver and then approved for delivery on the patient's regimen.

The Call Center provides support to caregivers, patients, family members and pharmacists. Call center users can create, retrieve, and manage medical alerts stored on the central monitoring station.

### Central Monitoring-Station Hardware

The central monitoring station consists of one or more highly customized servers running software applications that manage information for the health monitoring network.

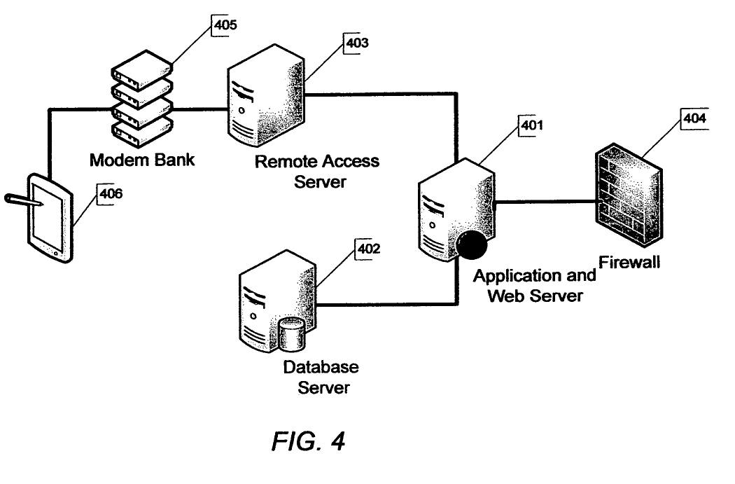

FIG. 4 illustrates three typical components for an instance of the central monitoring station on the Personal health monitoring network. Apparatus FIG. 4.401 illustrates an application server running Microsoft Windows Server 2003 and IIS (Internet Information Server). The application server executes specialized monitoring and control software. In one embodiment a Dell PowerEdge™2950 (Dell Computer Corporation, Round Rock, Tex.) serves as the application server. Apparatus FIG. 4.402 illustrates a database server used to store information for the central monitoring station. The database server runs Windows Server 2003 and SQL Server 2000. In this one embodiment a Dell PowerEdge™ 2950 server is utilized. Apparatus FIG. 4.403 illustrates a remote access server. The remote access server provides communications between the Personal Health Diaries and the central monitoring station. In this one embodiment a Dell PowerEdge™ 2950 running Windows Server 2003 is configured to run Microsoft's remote access server components. The remote access server components are interfaced to a modem bank (FIG. 4.405). In this one embodiment of the invention the modem bank consists of several Comtrol RocketModem IV™ cards (Comtrol Corporation, Maple Grove, Minn.). The apparatus in FIG. 4.406 illustrates the personal health diaries connecting to the remote access server through the modem bank using a dialup connection. In another embodiment of the invention the Personal Health Diaries connect to the central monitoring station through the firewall (FIG. 4.404). Using this feature Personal Health Diaries can be configured to connect to a local internet service provider or cellular data network which in turn utilizes the internet for information transfers. It should be recognized this feature allows patients around the world to cost effectively utilize the health monitoring network.

It will be recognized by those skilled in the art that the functions of the central monitoring station hardware can be accomplished using any number of hardware configurations and, in addition, that numerous other changes can be made in the embodiments described herein without departing from the scope and the spirit of the invention.

### Central Monitoring Station Software

The central monitoring station is an integrated telecommunication, database and application service which will provides the following functions:
Manages regimens for the personal health diaries.
Manages Patient Demographics
Creates Treatment Programs
Manages user profiles to provide security-for multiple concurrent users
Manages patient security to providing access to approved users
Reports the health status of patients
Reports the status of personal health diaries
Alerts Users of critical care conditions

The central monitoring station implements features to comply with all Federal regulations including user security, encryption and audit trails.

The central monitoring station database must store system information in a format which is efficient for the support of the health monitoring network. In one embodiment Microsoft SQL Server has been selected as the database engine. The organization of the data is derived from system use cases. It will be recognized by those skilled in the art that various data models can be accomplished and, in addition, that numerous other changes can be made to the database embodiments described herein without departing from the scope and the spirit of the invention.

The central monitoring station utilizes locale independent date and time formats. Date and Time values stored in the Database will be long Date Time values (date types). Date and Time values displayed on user interface screens and reports are displayed in a locale/time zone specific format. Date strings include a four digit year. US English time strings will be in 12 hour am/pm format designation.

The central monitoring station services provide a mechanism to set accurate time/date information on the servers and personal health diaries. The personal health diaries are programmed to synchronize time/date with the central monitoring station. The personal health diaries parse the date/time information updating their internal clock. In addition the central monitoring station is configured to automatically synchronize the time from well known time sources (e.g. time.nist.gov)

The central monitoring station provides a layered security implementation to protect the patient information. The central monitoring station implements the following security features:

### Hardware Security

The central monitoring station servers provide packet routing security through hardware based firewalls and routing. The firewall/router configuration insures that only TCP/IP traffic for HTTPS ports is routed to the servers. Routing for other services such is blocked.

### Operating System Level Security

The central monitoring stations routinely install operating system updates and security patches as recommended by the operating system manufacturer. The servers are locked down per operating system manufacturer's recommendations. The servers implement a minimal number of services required to deploy and maintain the system. The servers implement mechanisms to detect viruses and hacking attempts.

### Application Level Security

The central monitoring station's application provides role-based security. This role based model is the final layer in the system security. The backend application requires client login. The login interface is encrypted to protect user and patient information. Login attempts are noted in the audit logs. The application maintains a session based security system which prevents bypassing the logon screen. Attempts to bypass security will be redirected to the application login screen. The application will implement session based timeouts. Idle logins are terminated after a specified number of minutes of inactivity. Each screen displays the currently logged in user. The application based roles will define specific functionality available to the User.

### Database Structure

The elements of the central monitoring stations database can be defined in conceptual terms. The concepts define information groupings and business rules for the health monitoring network. The data for the central monitoring station is organized in a hierarchy of data entities. The tree below outlines the basic relationships:

```
Treatment Programs
    Regimens (Template Editions)
    Medications
    Alarms
    Questionnaires
      Questions
        Question Text
        Reponses
Participants
    Patients
    Diary Devices
    Regimen (Patient Editions)
      Medications
      Alarms
      Questionnaires
        Questions
          Question Text
          Reponses
```

The central monitoring stations software applications are implemented as object oriented applications.

### Software Structure

The central monitoring station software application class structure is depicted below:
Application Level
   User Interface Layer
   Business Application Layer (BAL)
   Data Application Layer (DAL)
   Common Application Layer (Common)

The Application Level is the main entry point for the software application. The user interface is directly implemented under this level. The user interface communicates with the Business application layer (the BAL). The BAL is responsible for implementation of all application functional logic; this includes management of regimens and component definitions, management of patient enrollment, transmission of regimens and multimedia content to the personal health diaries, reception of events from the personal health diaries, synchronization of real time clocks on the personal health diaries. The BAL Layer interfaces to the Data Application Layer (the DAL). In one embodiment of the invention the central monitoring station software implements web services using the Microsoft SOAP (Simple Object Access Protocol) to interface with the personal health diaries. The DAL is responsible for data storage/retrieval. The DAL Layer implements CRUDE (Create, Retrieve, Update, Delete, and Enumerate) data access functions. The Common Application Layer (Common) provides application utility functions which are shared across all the application layers. It will be recognized by those skilled in the art that various other software implementations can be implemented and, in addition, that numerous other changes can be made in the embodiments described herein without departing from the scope and the spirit of the invention.

### Additional Notices

In the interest of clarity and simplicity, the invention has been described in terms of a single patient. However, the skilled reader will appreciate that the invention may readily be employed in monitoring a plurality of patients. Similarly, the invention has been described in terms of a system having a single personal health diary, a single caregiver, a single pharmacist, and a single family member, but the skilled reader will appreciate that the invention may be implemented to support a plurality of any or all of those, either in conjunction with a single device or a plurality of devices. The skilled reader will further appreciate that variations of the functionalities described herein may in some embodiments be practiced at different locations or upon different hardware than that disclosed herein. As but one example, the records database and/or the behavioral model might be implemented at the pharmacy rather than at the patient's location, without departing from the scope of this invention.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments, of the invention. The various appearances “an embodiment,” “one embodiment,” or “some embodiments” are not necessarily all referring to the same embodiments.

If the specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

Those skilled in the art having the benefit of this disclosure will appreciate that many other variations from the foregoing description and drawings may be made within the scope of the present invention.

Indeed, the invention is not limited to the details described above. Rather, it is the following claims including any amendments thereto that define the scope of the invention.

What is claimed is:

1. An automated health monitoring system, comprising:
at least one central monitoring station;
at least one personal health diary, configured to be used by at least one patient, and configured to present individual medical regimens;
wherein said personal health diary is configured to collect said patient's subjective and physiological measurement data;
wherein said personal health diary establishes communications with said central monitoring station and communicates said subjective and physiological measurement data to said central monitoring station;
wherein said at least one central monitoring station further comprises means for configuring individual medical regimens for said at least one patient, and for communicating said regimens to said personal health diary; and
wherein said individual medical regimens are expressed as a markup language.

2. The automated health monitoring system of claim 1, wherein said at least one central monitoring station further comprises:
means for receiving time stamped user responses from said personal health diary;
means for establishment of communications with said personal health diary; and
means to make available medical histories for at least one patient.

3. The automated health monitoring system of claim 1, wherein said personal health diary presents periodic prompts and informational alerts to said at least one patient.

4. The automated health monitoring system of claim 1, wherein data collected from said personal health diary is used to establish an electronic medical record for the patient; and wherein said electronic medical record is used to generate health related alerts related to said patient's health condition.

5. The automated health monitoring system of claim 1, wherein said at least one central monitoring station further comprises:
means for creating and delivering periodic chronological medical records to the patient;
means for delivering said medical records through said personal health diary; and
means for delivering said medical records through related electronic and corporeal delivery mechanisms.

6. An electronic personal health diary apparatus, comprising:
means for presenting individual medical regimens to at least one patient;
means for collecting said patient's subjective and physiological measurement data;
means for establishing communications with a central monitoring station and communicating said subjective and physiological measurement data to said central monitoring station;
means for receiving and displaying individual medical regimens transmitted from said central monitoring station; and
wherein said individual medical regimens are expressed as a markup language.

7. The electronic personal health diary apparatus of claim 6, wherein said health diary apparatus is adapted to track personal medical conditions, and which further comprises periodically alerting a user of required medical care events and automatically measures and records related responses from the user.

8. The electronic personal health diary apparatus of claim 6, wherein said health diary apparatus is programmable, and adapted for use in a patient's home, and further comprises a visual display, a patient input mechanism, and at least one associated health sensor.

9. The electronic personal health diary apparatus of claim 8, further comprising a scheduler mechanism that produces audible alerts and presents prompts to a patient via said visual display.

10. The electronic personal health diary apparatus of claim 6, further comprising:
a time base which directs delivery of periodic prompts and informational alerts for purposes of eliciting user responses;
periodically logging time and recorded data of device operational events;
a time base which tracks a time of said recorded data;
a data storage system which logs said time and said recorded data to build a sequential event history of said user;
wherein said data storage system logs said time and said recorded data to build a sequential event history of said device operation.

11. The electronic personal health diary apparatus of claim 10, wherein said data storage system stores said individual medical regimens for said user.

12. The electronic personal health diary apparatus of claim 6, wherein said apparatus is remotely configured with said individual medical regimens, which comprise medical directives individually tailored to said patient's lifestyle.

13. The electronic personal health diary apparatus of claim 6, wherein said individual medical regimen comprises a plurality of subjective health questionnaires.

14. The electronic personal health diary apparatus of claim 6, wherein said individual medical regimen comprises physiological measurement prompts, which comprise blood pressure, pulse rate, blood glucose, blood oxygen saturation, weight, temperature, and/or pulmonary function, comprising respiratory rate and depth.

15. The electronic personal health diary apparatus of claim 6, wherein said individual medical regimen comprises medication dosing reminders.

16. The electronic personal health diary apparatus of claim 6, wherein said individual medical regimen comprises educational articles relating to said patient's medical condition.

17. The electronic personal health diary apparatus of claim 6, wherein said individual medical regimen comprises news articles relating to said patient's medical condition.

18. The electronic personal health diary apparatus of claim 6, wherein said individual medical regimen comprises personal multimedia content intended to inspire said patient to follow medical advice, wherein said multimedia content comprises calendar scheduling, educational videos, family pictures, family video messages, voice messages, electronic letters, and/or electronic greeting cards.

19. The electronic personal health diary apparatus of claim 6, wherein said markup language comprises:
a set of descriptive tags defining a structural beginning and end of said health regimen; and
a set of structural beginning and ending tags for each element of said health regimen.

20. The electronic personal health diary apparatus of claim 19, wherein said markup language further comprises:
a collection of attributes within said beginning tags which further characterize a behavior of an associated tag; and
a plurality of tags, which define:
health regimen event types;
calendar scheduling of events;
intraday timing of events;
medication dosages;
health related education articles;
health related news articles;
health related questionnaires;
health related physiological measurements; and/or
health related personal multimedia content.

21. The electronic personal health diary apparatus of claim 10, wherein said markup language comprises:
a set of descriptive tags defining a structural beginning and end of said recorded data; and
a set of structural beginning and ending tags for each element of said recorded data.

22. The electronic personal health diary apparatus of claim 21, wherein said markup language further comprises:
a collection of attributes within said beginning tags which further characterize a behavior of an associated tag;
at least one set of tags which define the specific health regimen associated with the recorded data;
at least one set of tags which define the date and time of the recorded data;
a plurality of tags, which define:
specific events within the recorded data;
patient responses within the recorded data;
physiological measurement within the recorded data; and/or
operational events of the personal health dairy within the recorded data.

23. The electronic personal health diary apparatus of claim 6, further comprising:
means for transferring individual medical regimens from a central monitoring station;
means for storing said individual medical regimens; and
means for retaining all configuration and recorded data in event of a power outage or battery failure.

24. The electronic personal health diary apparatus of claim 6, further comprising:
means for synchronizing time and date with a central time source; and
means for permitting patients to adjust time zone and daylight savings to their current location; and/or
means for permitting caregivers to remotely adjust time zone and daylight savings for a patients current location.

25. The electronic personal health diary apparatus of claim 6, further comprising means for permitting said patient to adjust operating language.

26. The electronic personal health diary apparatus of claim 6, further comprising means for permitting incoming phone calls from caregivers and family members.

27. The automated health monitoring system of claim 1, wherein said at least one central monitoring station further comprises:
means for generating alerts to family members of said patient.

28. The automated health monitoring system of claim 1, wherein said at least one central monitoring station further comprises:
means for accepting event requests from family members of said patient, for delivery of patient specific content to said patient via said personal health diary.

29. The electronic personal health diary apparatus of claim 19, further comprising a regimen parser which ignores new unknown tags.

30. The electronic personal health diary apparatus of claim 21, further comprising a regimen parser which ignores new unknown tags.

31. A method for automated health monitoring, comprising the steps of:
providing at least one central monitoring station;
providing at least one personal health diary, configured to be used by at least one patient, and configured to present individual medical regimens;
collecting said patient's subjective and physiological measurement data using said personal health diary;
establishing communications with said central monitoring station and communicating said subjective and physiological measurement data to said central monitoring station, using said personal health diary;
configuring individual medical regimens for said at least one patient through said central monitoring station, and communicating said regimens to said personal health diary; and expressing said individual medical regimens as a markup language.

32. The method for automated health monitoring of claim 31, wherein said at least one central monitoring station further comprises:
receiving time stamped user responses from said personal health diary;
establishing communications with said personal health diary; and
making available medical histories for at least one patient.
